(19) United States
(12) Patent Application Publication
(10) Pub. No.: US 2023/0245316 A1
(43) Pub. Date: Aug. 3, 2023

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventors: Keigo NAKAMURA, Tokyo (JP); Jun MASUMOTO, Tokyo (JP)

(73) Assignee: FUJIFILM Corporation, Tokyo (JP)

(21) Appl. No.: 18/161,813

(22) Filed: Jan. 30, 2023

(30) Foreign Application Priority Data
Feb. 3, 2022 (JP) 2022-015964

(51) Int. Cl. G06T 7/00; G06T 7/11; G06V 10/26; G06V 30/412; G16H 50/20

(52) U.S. Cl. CPC G06T 7/0016; G06T 7/11; G06V 10/26; G06V 30/412; G16H 50/20; G06T 2207/30064; G06T 2207/30096; G06T 2207/30016; G06T 2207/30084; G06T 2207/30056; G06T 2207/30176; G06T 2207/10081; G06V 2201/032

(57) ABSTRACT

An information processing apparatus comprising at least one processor, wherein the at least one processor is configured to: acquire a document describing a subject; extract document finding information indicating a finding of the subject included in the document; and specify a finding extraction process for extracting image finding information indicating the finding indicated by the document finding information from a first image obtained by imaging the subject, among a plurality of types of finding extraction processes for extracting image finding information indicating a plurality of different types of findings that are able to be included in the first image.

### CROSS-REFERENCE TO RELATED APPLICATIONS

This application claims priority from Japanese Application No. 2022-015964, filed on Feb. 3, 2022, the entire disclosure of which is incorporated herein by reference.

### BACKGROUND

### Technical Field

The present disclosure relates to an information processing apparatus, an information processing method, and an information processing program.

### Related Art

In the related art, image diagnosis is performed using medical images obtained by imaging apparatuses such as computed tomography (CT) apparatuses and magnetic resonance imaging (MRI) apparatuses. In addition, medical images are analyzed via computer aided detection/diagnosis (CAD) using a discriminator in which learning is performed by deep learning or the like, and regions of interest including structures, lesions, and the like included in the medical images are detected and/or diagnosed. The medical images and analysis results via CAD are transmitted to a terminal of a healthcare professional such as a radiologist who interprets the medical images. The healthcare professional such as a radiologist interprets the medical image by referring to the medical image and analysis result using his or her own terminal and creates an interpretation report.

That is, the analysis result via CAD is described in the interpretation report after being checked by the radiologist. For example, JP2013-041428A discloses a technique for supporting a doctor in reviewing an interpretation finding by comparing a CAD finding obtained by analyzing examination data of a subject with the interpretation finding by the doctor on the examination data.

Incidentally, it is known that a region of interest in a medical image has significant physical features. For example, in a CT image of the brain, cerebral hemorrhage is suspected in a region of a relatively white mass compared to the surroundings, and cerebral infarction is suspected in a region of a relatively black mass compared to the surroundings. Therefore, in a case where the radiologist interprets the medical image, if the visibility of the region of interest in the medical image can be improved by performing the finding extraction process according to the type of the region of interest, the interpretation can be facilitated.

On the other hand, with the recent improvement in the performance of the imaging apparatus and the performance of the CAD, the types of regions of interest that can be detected by the CAD and the types of the finding extraction processes corresponding to the types of the regions of interest are increasing. It may take time and effort for radiologists to execute the finding extraction process for checking the desired region of interest in the medical image through trial and error.

### SUMMARY

The present disclosure provides an information processing apparatus, an information processing method, and an information processing program capable of supporting interpretation of images.

According to a first aspect of the present disclosure, there is provided an information processing apparatus comprising at least one processor, in which the processor is configured to: acquire a document describing a subject; extract document finding information indicating a finding of the subject included in the document; and specify a finding extraction process for extracting image finding information indicating the finding indicated by the document finding information from a first image obtained by imaging the subject, among a plurality of types of finding extraction processes for extracting image finding information indicating a plurality of different types of findings that are able to be included in the first image.

In the first aspect, the processor may be configured to: acquire the first image; and extract the image finding information indicating at least one type of findings included in the first image by executing the plurality of types of finding extraction processes on the first image.

In the first aspect, the processor may be configured to associate an extraction result of the document finding information for the same region of interest with an extraction result of the image finding information.

In the first aspect, the processor may be configured to associate an extraction result of the document finding information with an extraction result of the image finding information obtained by executing the finding extraction process of the same type as the finding extraction process specified for the document finding information on the first image.

In the first aspect, the processor may be configured to present a finding included in both the extraction result of the document finding information and the extraction result of the image finding information, and a finding included in any one of the extraction result of the document finding information and the extraction result of the image finding information in an identifiable manner.

In the first aspect, the processor may be configured to make presentation indicating a possibility of omission of extraction by the finding extraction process for a finding that is included in the extraction result of the document finding information and is not included in the extraction result of the image finding information.

In the first aspect, the document may be a document describing a second image obtained by imaging the subject at an imaging point in time prior to an imaging point in time of the first image, and the processor may be configured to make presentation indicating being followed up for a finding that is included in the extraction result of the document finding information and is included in the extraction result of the image finding information.

In the first aspect, the document may be a document describing a second image obtained by imaging the subject at an imaging point in time prior to an imaging point in time of the first image, and the processor may be configured to make presentation indicating a new finding for a finding that is not included in the extraction result of the document finding information and is included in the extraction result of the image finding information.

In the first aspect, the document may be a document describing a second image obtained by imaging the subject at an imaging point in time prior to an imaging point in time of the first image.

In the first aspect, the first image may be a medical image, the document finding information and the image finding information may be information indicating at least one of a name, a property, a measured value, a position, or an estimated disease name related to a region of interest included in the medical image, and the region of interest may be at least one of a region of a structure included in the medical image or a region of an abnormal shadow included in the medical image.

According to a second aspect of the present disclosure, there is provided an information processing method comprising: acquiring a document describing a subject; extracting document finding information indicating a finding of the subject included in the document; and specifying a finding extraction process for extracting image finding information indicating the finding indicated by the document finding information from a first image obtained by imaging the subject, among a plurality of types of finding extraction processes for extracting image finding information indicating a plurality of different types of findings that are able to be included in the first image.

According to a third aspect of the present disclosure, there is provided an information processing program for causing a computer to execute a process comprising: acquiring a document describing a subject; extracting document finding information indicating a finding of the subject included in the document; and specifying a finding extraction process for extracting image finding information indicating the finding indicated by the document finding information from a first image obtained by imaging the subject, among a plurality of types of finding extraction processes for extracting image finding information indicating a plurality of different types of findings that are able to be included in the first image.

With the information processing apparatus, the information processing method, and the information processing program according to the aspects of the present disclosure, it is possible to support the interpretation of images.

### BRIEF DESCRIPTION OF THE DRAWINGS

FIG. 1 is a diagram showing an example of a schematic configuration of an information processing system.

FIG. 2 is a diagram showing an example of a medical image.

FIG. 3 is a diagram showing an example of a medical image.

FIG. 4 is a block diagram showing an example of a hardware configuration of an information processing apparatus.

FIG. 5 is a block diagram showing an example of a functional configuration of the information processing apparatus.

FIG. 6 is a diagram showing an example of an interpretation report.

FIG. 7 is a diagram showing an example of document finding information.

FIG. 8 is a diagram showing an example of a finding extraction process.

FIG. 9 is a diagram showing an example of a screen displayed on a display.

FIG. 10 is a flowchart showing an example of first information processing.

FIG. 11 is a diagram showing an example of image finding information.

FIG. 12 is a diagram showing an example of a result of associating document finding information with image finding information.

FIG. 13 is a diagram showing an example of a pattern based on document finding information and image finding information.

FIG. 14 is a diagram showing an example of a screen displayed on a display.

FIG. 15 is a flowchart showing an example of second information processing.

FIG. 16 is a diagram showing an example of an interpretation report.

FIG. 17 is a diagram showing an example of document finding information.

FIG. 18 is a diagram showing an example of image finding information.

FIG. 19 is a diagram showing an example of a result of associating document finding information with image finding information.

### DETAILED DESCRIPTION

Each embodiment of the present disclosure will be described below with reference to the drawings.

### First Embodiment

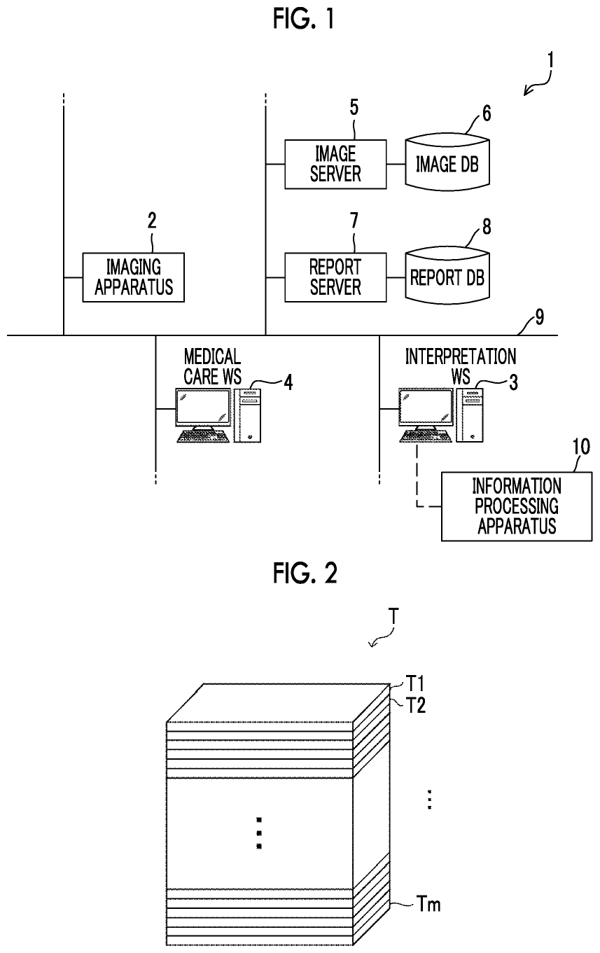

First, a configuration of an information processing system 1 to which an information processing apparatus of the present disclosure is applied will be described. FIG. 1 is a diagram showing a schematic configuration of the information processing system 1. The information processing system 1 shown in FIG. 1 performs imaging of an examination target part of a subject and storing of a medical image acquired by the imaging based on an examination order from a doctor in a medical department using a known ordering system. In addition, the information processing system 1 performs an interpretation work of a medical image and creation of an interpretation report by a radiologist and viewing of the interpretation report by a doctor of a medical department that is a request source.

As shown in FIG. 1, the information processing system 1 includes an imaging apparatus 2, an interpretation work station (WS) 3 that is an interpretation terminal, a medical care WS 4, an image server 5, an image database (DB) 6, a report server 7, and a report DB 8. The imaging apparatus 2, the interpretation WS 3, the medical care WS 4, the image server 5, the image DB 6, the report server 7, and the report DB 8 are connected to each other via a wired or wireless network 9 in a communicable state.

Each apparatus is a computer on which an application program for causing each apparatus to function as a component of the information processing system 1 is installed. The application program may be recorded on, for example, a recording medium, such as a digital versatile disc (DVD) or a compact disc read only memory (CD-ROM), and distributed, and be installed on the computer from the recording medium. In addition, the application program may be stored in, for example, a storage apparatus of a server computer connected to the network 9 or in a network storage in a state in which it can be accessed from the outside, and be downloaded and installed on the computer in response to a request.

The imaging apparatus 2 is an apparatus (modality) that generates a medical image T showing a diagnosis target part of the subject by imaging the diagnosis target part. Specifically, examples of the imaging apparatus 2 include a simple X-ray imaging apparatus, a CT apparatus, an MRI apparatus, a positron emission tomography (PET) apparatus, and the like. The medical image generated by the imaging apparatus 2 is transmitted to the image server 5 and is saved in the image DB 6.

The interpretation WS 3 is a computer used by, for example, a healthcare professional such as a radiologist of a radiology department to interpret a medical image and to create an interpretation report, and encompasses an information processing apparatus 10 according to the present embodiment. In the interpretation WS 3, a viewing request for a medical image to the image server 5, various image processing for the medical image received from the image server 5, display of the medical image, and input reception of a sentence regarding the medical image are performed. In the interpretation WS 3, an analysis process for medical images, support for creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by, for example, a healthcare professional such as a doctor in a medical department to observe a medical image in detail, view an interpretation report, create an electronic medical record, and the like, and is configured to include a processing apparatus, a display apparatus such as a display, and an input apparatus such as a keyboard and a mouse. In the medical care WS 4, a viewing request for the medical image to the image server 5, display of the medical image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 is connected to the image DB 6. The connection form between the image server 5 and the image DB 6 is not particularly limited, and may be a form connected by a data bus, or a form connected to each other via a network such as a network attached storage (NAS) and a storage area network (SAN).

The image DB 6 is realized by, for example, a storage medium such as a hard disk drive (HDD), a solid state drive (SSD), and a flash memory. In the image DB 6, the medical image acquired by the imaging apparatus 2 and accessory information attached to the medical image are registered in association with each other.

The accessory information may include, for example, identification information such as an image identification (ID) for identifying a medical image, a tomographic ID assigned to each tomographic image included in the medical image, a subject ID for identifying a subject, and an examination ID for identifying an examination. In addition, the accessory information may include, for example, information related to imaging such as an imaging method, an imaging condition, and an imaging date and time related to imaging of a medical image. The “imaging method” and “imaging condition” are, for example, a type of the imaging apparatus 2, an imaging part, an imaging protocol, an imaging sequence, an imaging method, the presence or absence of use of a contrast medium, a slice thickness in tomographic imaging, and the like. In addition, the accessory information may include information related to the subject such as the name, age, and gender of the subject.

In a case where the image server 5 receives a request to register a medical image from the imaging apparatus 2, the image server 5 prepares the medical image in a format for a database and registers the medical image in the image DB 6. In addition, in a case where the viewing request from the interpretation WS 3 and the medical care WS 4 is received, the image server 5 searches for a medical image registered in the image DB 6 and transmits the searched for medical image to the interpretation WS 3 and to the medical care WS 4 that are viewing request sources.

The report server 7 is a general-purpose computer on which a software program that provides a function of a database management system is installed. The report server 7 is connected to the report DB 8. The connection form between the report server 7 and the report DB 8 is not particularly limited, and may be a form connected by a data bus or a form connected via a network such as a NAS and a SAN.

The report DB 8 is realized by, for example, a storage medium such as an HDD, an SSD, and a flash memory. In the report DB 8, an interpretation report created in the interpretation WS 3 is registered.

Further, in a case where the report server 7 receives a request to register the interpretation report from the interpretation WS 3, the report server 7 prepares the interpretation report in a format for a database and registers the interpretation report in the report DB 8. Further, in a case where the report server 7 receives the viewing request for the interpretation report from the interpretation WS 3 and the medical care WS 4, the report server 7 searches for the interpretation report registered in the report DB 8, and transmits the searched for interpretation report to the interpretation WS 3 and to the medical care WS 4 that are viewing request sources.

The network 9 is, for example, a network such as a local area network (LAN) and a wide area network (WAN). The imaging apparatus 2, the interpretation WS 3, the medical care WS 4, the image server 5, the image DB 6, the report server 7, and the report DB 8 included in the information processing system 1 may be disposed in the same medical institution, or may be disposed in different medical institutions or the like. Further, the number of each apparatus of the imaging apparatus 2, the interpretation WS 3, the medical care WS 4, the image server 5, the image DB 6, the report server 7, and the report DB 8 is not limited to the number shown in FIG. 1, and each apparatus may be composed of a plurality of apparatuses having the same functions.

FIG. 2 is a diagram schematically showing an example of a medical image acquired by the imaging apparatus 2. The medical image T shown in FIG. 2 is, for example, a CT image consisting of a plurality of tomographic images T1 to Tm (m is 2 or more) representing tomographic planes from the chest to the lumbar region of one subject (human body). The medical image T and the tomographic images T1 to Tm are examples of a first image and a second image of the present disclosure.

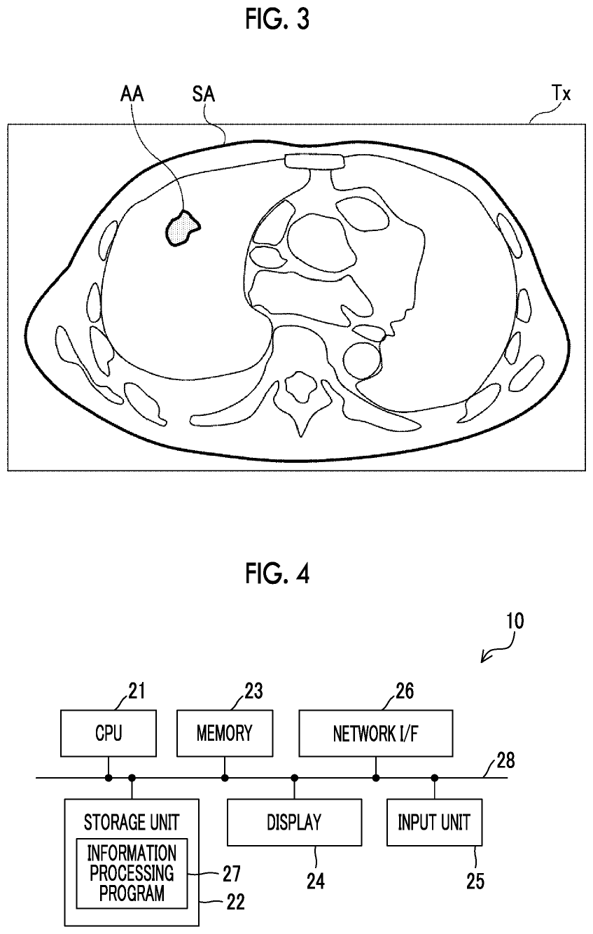

FIG. 3 is a diagram schematically showing an example of one tomographic image Tx out of the plurality of tomographic images T1 to Tm. The tomographic image Tx shown in FIG. 3 represents a tomographic plane including a lung. Each of the tomographic images T1 to Tm may include a region SA of a structure showing various organs of the human body (for example, lungs, livers, and the like), various tissues constituting various organs (for example, blood vessels, nerves, muscles, and the like), and the like. In addition, each tomographic image may include lesions (for example, nodules, tumors, injuries, defects, inflammation, and the like), and a region AA of an abnormal shadow showing regions obscured by imaging. In the tomographic image Tx shown in FIG. 3, the lung region is the region SA of the structure, and the nodule region is the region AA of the abnormal shadow. Hereinafter, at least one of the region SA of the structure or the region AA of the abnormal shadow is referred to as a “region of interest”. Note that one tomographic image may include a plurality of regions of interest.

Next, the information processing apparatus 10 will be described. The information processing apparatus 10 according to the present embodiment has a function of supporting the user in interpreting a medical image. As described above, the information processing apparatus 10 is encompassed in the interpretation WS 3.

First, with reference to FIG. 4, an example of a hardware configuration of the information processing apparatus 10 according to the present embodiment will be described. As shown in FIG. 4, the information processing apparatus 10 includes a central processing unit (CPU) 21, a non-volatile storage unit 22, and a memory 23 as a temporary storage area. Further, the information processing apparatus 10 includes a display 24 such as a liquid crystal display, an input unit 25 such as a keyboard and a mouse, and a network interface (I/F) 26. The network I/F 26 is connected to the network 9 and performs wired or wireless communication. The CPU 21, the storage unit 22, the memory 23, the display 24, the input unit 25, and the network I/F 26 are connected to each other via a bus 28 such as a system bus and a control bus so that various types of information can be exchanged.

The storage unit 22 is realized by, for example, a storage medium such as an HDD, an SSD, and a flash memory. An information processing program 27 in the information processing apparatus 10 is stored in the storage unit 22. The CPU 21 reads out the information processing program 27 from the storage unit 22, loads the read-out program into the memory 23, and executes the loaded information processing program 27. The CPU 21 is an example of a processor of the present disclosure. As the information processing apparatus 10, for example, a personal computer, a server computer, a smartphone, a tablet terminal, a wearable terminal, or the like can be appropriately applied.

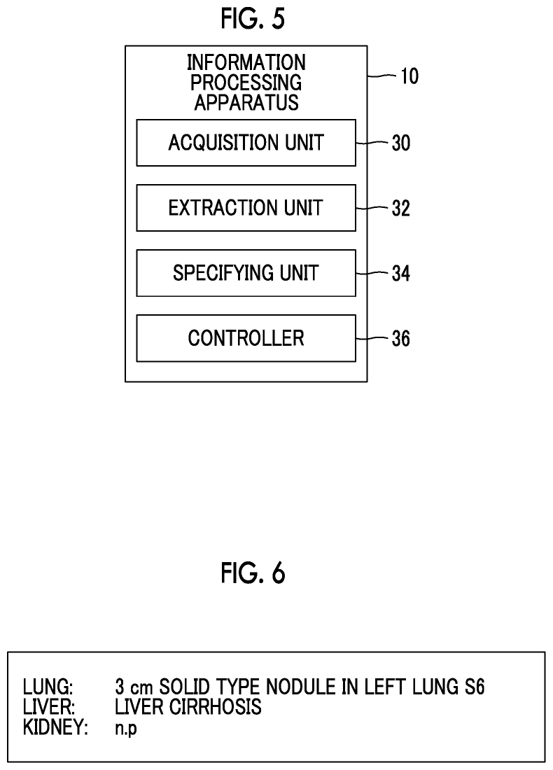

Next, with reference to FIG. 5, an example of a functional configuration of the information processing apparatus 10 according to the present embodiment will be described. As shown in FIG. 5, the information processing apparatus 10 includes an acquisition unit 30, an extraction unit 32, a specifying unit 34, and a controller 36. As the CPU 21 executes the information processing program 27, the CPU 21 functions as the acquisition unit 30, the extraction unit 32, the specifying unit 34, and the controller 36.

The acquisition unit 30 acquires an interpretation report describing the subject from the report server 7. FIG. 6 shows an example of an interpretation report. The interpretation report shown in FIG. 6 includes a description showing the findings about the lung, a description showing the findings about the liver, and a description showing no particularity (n.p: not particular) about the kidney. An interpretation report is an example of a document of the present disclosure.

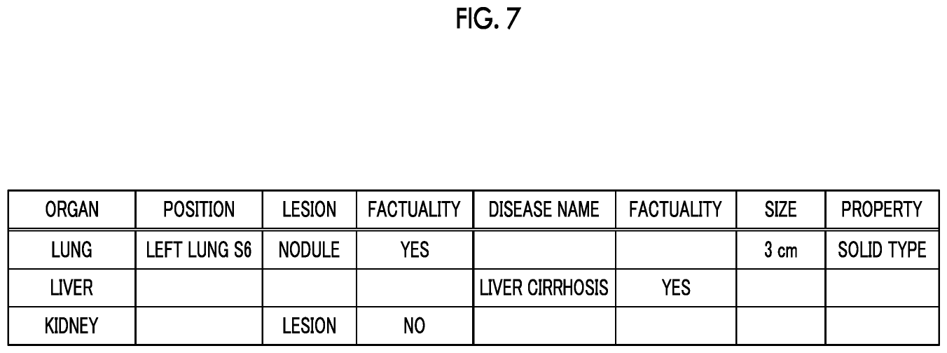

The extraction unit 32 extracts document finding information indicating a finding of the subject included in the interpretation report acquired by the acquisition unit 30. FIG. 7 shows document finding information extracted from the interpretation report of FIG. 6. As shown in FIG. 7, the document finding information is, for example, information indicating at least one of a name (type), a property, a measured value, a position, or an estimated disease name related to a region of interest included in a medical image.

Examples of names (types) include the names of structures such as “lung” and “liver”, and the names of abnormal shadows such as “nodule”. The property mainly mean the features of abnormal shadows. For example, in the case of a lung nodule, findings indicating absorption values such as “solid type” and “frosted glass type”, margin shapes such as “clear/unclear”, “smooth/irregular”, “spicula”, “lobulation”, and “serration”, and an overall shape such as “round shape” and “irregular shape” can be mentioned. In addition, for example, there are findings regarding the relationship with surrounding tissues such as “pleural contact” and “pleural invagination”, and the presence or absence of contrast enhancement, washout, and the like.

Examples of the measured value include a value that can be quantitatively measured from a medical image, and examples thereof include a size (a major axis, a minor axis, a volume, and the like), a CT value whose unit is HU, the number of regions of interest in a case where there are a plurality of regions of interest, and a distance between regions of interest. Further, the measured value may be replaced with a qualitative expression such as “large/small” or “more/less”. The position means an anatomical position, a position in a medical image, and a relative positional relationship with other regions of interest such as “inside”, “margin”, and “periphery”. The anatomical position may be indicated by an organ name such as “lung” and “liver”, and may be expressed in terms of subdivided lungs such as “right lung”, “upper lobe”, and apical segment (“S1”). The estimated disease name is an evaluation result estimated by the extraction unit 32 based on the abnormal shadow, and, for example, the disease name such as “liver cirrhosis”, “cancer”, and “inflammation” and the evaluation result such as “negative/positive”, “benign/malignant”, and “mild/severe” regarding disease names and properties can be mentioned.

Specifically, the extraction unit 32 may structure each sentence in the interpretation report using a known natural language analysis. For example, the document finding information included in the interpretation report may be extracted by extracting words in the interpretation report and collating the extracted words with a dictionary in which the various types of document finding information with words are associated in advance. The dictionary may be stored in advance in, for example, the storage unit 22.

In addition, it is preferable that the extraction unit 32 specifies the factuality of the word corresponding to the document finding information based on the arrangement of the words. The “factuality” is information indicating whether the finding is found or not, and the degree of certainty thereof and the like. This is because the interpretation report may include not only the findings that are clearly found from the medical image, but also the findings that are suspected to have a low degree of certainty or are not found from the medical image. For example, for a lung nodule, the presence or absence of “calcification” may be used for diagnosing the degree of severity, and the interpretation report may intentionally state that “no calcification is found”.

Here, a plurality of types of finding extraction processes for extracting image finding information indicating a plurality of different types of findings that may be included in the medical image will be described. The image finding information is, for example, information indicating at least one of a name (type), a property, a measured value, a position, or an estimated disease name related to a region of interest included in a medical image. The details of the various types of information indicated by the image finding information are the same as the details of the various types of information indicated by the document finding information described above, and thus the description thereof will be omitted.

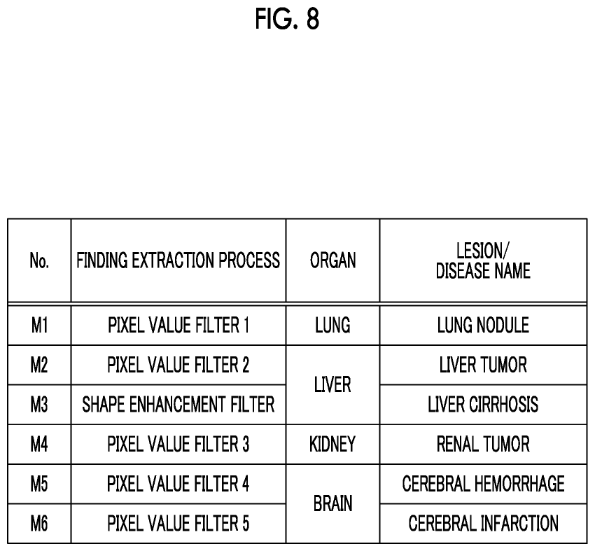

FIG. 8 shows a list of a plurality of types of finding extraction processes M1 to M6 for extracting various types of image finding information from a medical image. As shown in FIG. 8, the finding extraction processes M1 to M6 have different organs, lesions, and/or disease names to be extracted. By applying the finding extraction processes M1 to M6 to the medical image, image finding information related to the organ, the lesion, and/or the disease name to be extracted is extracted. The correspondence relationship between the finding extraction processes M1 to M6 and the organ, the lesion, and/or the disease name to be extracted is stored in advance in the storage unit 22 as, for example, a table.

The finding extraction processes M1, M2, and M4 to M6 (“pixel value filters 1 to 5”) are pixel value filters having different threshold values, such as a high-pass filter and a low-pass filter. For example, in a CT image of the brain, cerebral hemorrhage is suspected in a region of a relatively white mass compared to the surroundings, and cerebral infarction is suspected in a region of a relatively black mass compared to the surroundings. Therefore, for example, in a case where a region of a relatively white mass compared to the surroundings is detected as a result of applying the finding extraction process M5 (“pixel value filter 4”) to the medical image of the brain, image finding information indicating the presence of cerebral hemorrhage can be extracted. On the other hand, in a case where a region of a relatively black mass compared to the surroundings is detected as a result of applying the finding extraction process M6 (“pixel value filter 5”) to the medical image of the brain, image finding information indicating the presence of cerebral infarction can be extracted.

The finding extraction process M3 (“shape enhancement filter”) is a known shape enhancement filter such as an edge detection filter. For example, in a CT image of the liver, liver cirrhosis is suspected in a case where the liver has an uneven shape with irregular margins. Therefore, for example, in a case where a region of an uneven shape with irregular margins is detected as a result of applying the finding extraction process M3 (“shape enhancement filter”) to the medical image of the liver, image finding information indicating liver cirrhosis can be extracted.

In addition, as the finding extraction process, for example, a trained model such as convolutional neural network (CNN), which has been trained in advance so that the input is a medical image and the output is image finding information extracted from the medical image, may be used. This trained model is, for example, a model trained by machine learning using, as training data, a combination of a medical image in which a region of interest (that is, a region having a predetermined physical feature) is known and image finding information indicated by a region of interest included in the medical image. The “region having a physical feature” includes, for example, a region in a range in which the pixel value is preset (for example, a region in which the pixel value is relatively white/black mass compared to the surroundings) and a region having a preset shape.

For example, instead of the finding extraction process M1 (“pixel value filter 1”), a trained model, which has been trained in advance so that the input is a medical image of the lung, and the output is an image finding information indicating the properties, measured values, positions, estimated disease names, and the like of the lung nodule extracted from the medical image, may be used. Further, for example, instead of the finding extraction process M3 (“shape enhancement filter”), a trained model, which has been trained in advance so that the input is a medical image of the liver, and the output is an image finding information indicating the properties, measured values, positions, estimated disease names (for example, liver cirrhosis), and the like of the liver extracted from the medical image, may be used. Further, for example, instead of the finding extraction process M5 (“pixel value filter 4”), a trained model, which has been trained in advance so that the input is a medical image of the brain, and the output is an image finding information indicating the properties, measured values, positions, estimated disease names, and the like of the cerebral hemorrhage extracted from the medical image, may be used.

Further, image finding information may be extracted from the medical image by combining a plurality of trained models. For example, instead of the finding extraction process M1 (“pixel value filter 1”), a first trained model in which the input is a medical image of the lung and the output is a region of the lung nodule extracted from the medical image and a second trained model in which the input is the region of the lung nodule extracted from the medical image and the output is the image finding information of the region of the lung nodule may be used in combination.

The specifying unit 34 specifies a finding extraction process for extracting, from a medical image, image finding information indicating a finding indicated by the document finding information extracted from the interpretation report by the extraction unit 32, among the plurality of types of finding extraction processes determined in advance. Specifically, the specifying unit 34 specifies the corresponding finding extraction process by collating the document finding information (see FIG. 7) extracted from the interpretation report with a table in which a correspondence relationship between the finding extraction process and the organ, lesion, and/or disease name to be extracted is defined (see FIG. 8).

In the examples of FIGS. 6 to 8, the specifying unit 34 specifies the “pixel value filter 1” (finding extraction process M1) as the finding extraction process for extracting the image finding information indicating the “lung nodule” corresponding to the document finding information indicating the “nodule” of the “lung”. In addition, the specifying unit 34 specifies the “shape enhancement filter” (finding extraction process M3) as the finding extraction process for extracting the image finding information indicating the “liver cirrhosis” corresponding to the document finding information indicating the “liver cirrhosis” of the “liver”.

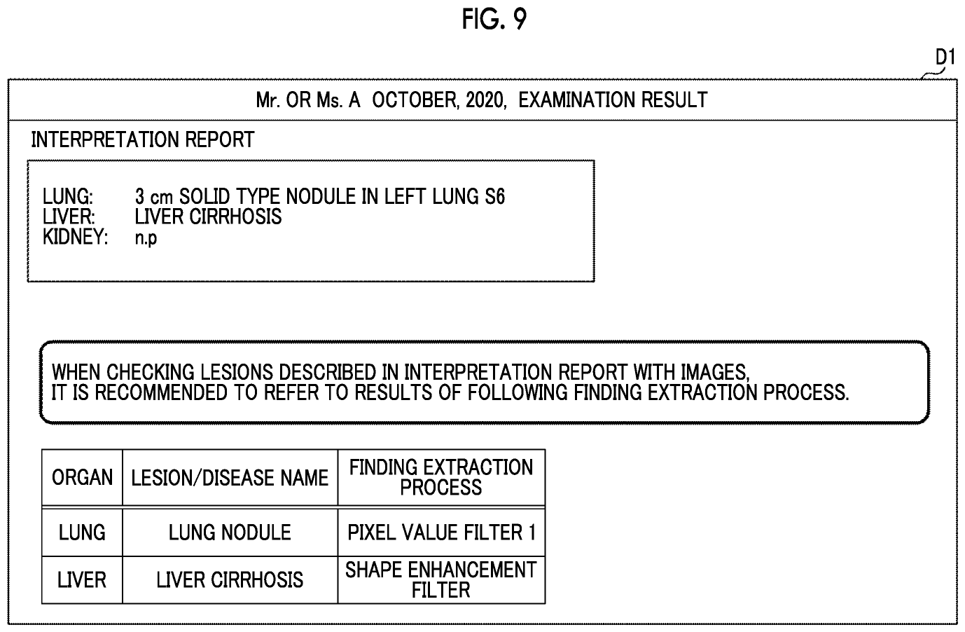

The controller 36 presents the finding extraction process specified by the specifying unit 34 with respect to the document finding information extracted from the interpretation report by the extraction unit 32. FIG. 9 is an example of a screen D1 displayed on the display 24 by the controller 36. The screen D1 includes an interpretation report acquired by the acquisition unit 30. In addition, the document finding information specified by the specifying unit 34 and the finding extraction process are presented in association with each other.

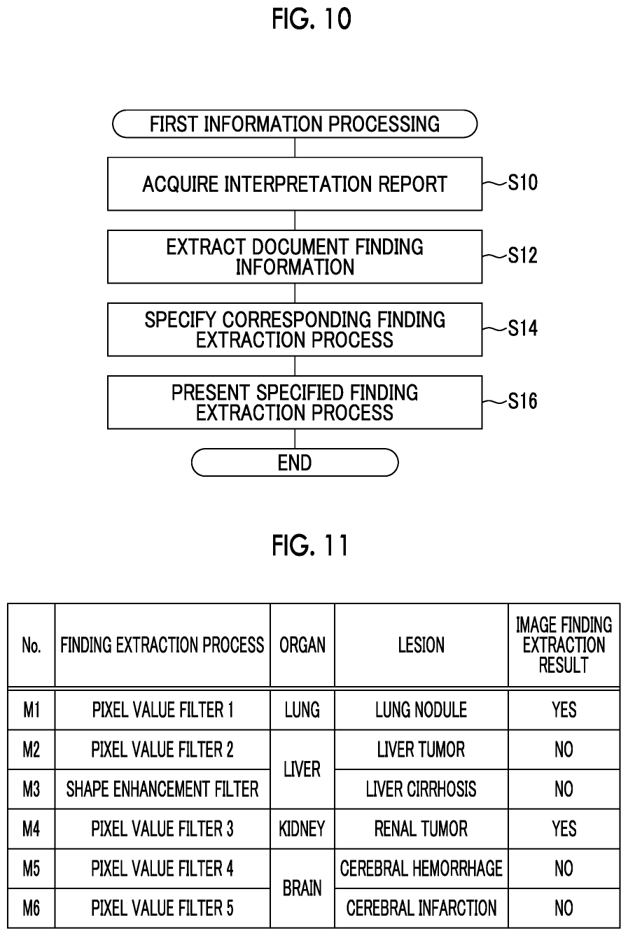

Next, with reference to FIG. 10, operations of the information processing apparatus 10 according to the present embodiment will be described. In the information processing apparatus 10, the CPU 21 executes the information processing program 27, and thus first information processing shown in FIG. 10 is executed. The first information processing is executed, for example, in a case where the user gives an instruction to start execution via the input unit 25.

In Step S10, the acquisition unit 30 acquires the interpretation report from the report server 7. In Step S12, the extraction unit 32 extracts the document finding information included in the interpretation report acquired in Step S10. In Step S14, the specifying unit 34 specifies the finding extraction process for extracting the image finding information indicating the finding indicated by the document finding information extracted in Step S12, among the plurality of types of finding extraction processes determined in advance. In Step S16, the controller 36 presents the finding extraction process specified in Step S14, and ends the first information processing.

As described above, the information processing apparatus 10 according to one aspect of the present disclosure comprises at least one processor, and the processor acquires a document describing a subject, extracts document finding information indicating a finding of the subject included in the document, and specifies a finding extraction process for extracting image finding information indicating the finding indicated by the document finding information from a first image obtained by imaging the subject, among a plurality of types of finding extraction processes for extracting image finding information indicating a plurality of different types of findings that are able to be included in the first image.

That is, with the information processing apparatus 10 according to the present embodiment, it is possible to specify an appropriate finding extraction process in a case where the findings described in the interpretation report are interpreted from the medical image. Therefore, for example, since it is possible to grasp an appropriate finding extraction process for the organ and lesion for which the interpretation is desired in a case where the reader of the interpretation report checks the medical image, or in a case where the radiologist redoes the interpretation, it is possible to support the interpretation of the medical image.

In addition, for example, in a case of performing follow-up observation on the same subject, interpretation of a medical image at a current point in time may be performed with reference to an interpretation report created at a past point in time. In other words, in the interpretation work of the medical image at the current point in time, the interpretation may be performed while searching for the findings described in the interpretation report created at the past point in time. With the information processing apparatus 10 according to the present embodiment, since an appropriate finding extraction process can be specified in a case where the findings described in the interpretation report created at the past point in time are checked in the medical image at the current point in time, it is possible to support the interpretation of the medical image. That is, the “document” to be processed by the information processing apparatus 10 according to the present embodiment may be a document describing a past medical image obtained by imaging the subject at an imaging point in time prior to an imaging point in time of the medical image to be subjected to the finding extraction process.

### Second Embodiment

The information processing apparatus 10 according to the present embodiment supports the user in interpreting the medical image, specializing in a case where the medical image at the current point in time is interpreted with reference to the interpretation report created at the past point in time. Hereinafter, the information processing apparatus 10 according to the second embodiment will be described, but the same configurations and functions as those of the first embodiment will be omitted as appropriate.

The acquisition unit 30 acquires a medical image at a current point in time (hereinafter referred to as “current image”) from the image server 5. Further, the acquisition unit 30 acquires an interpretation report describing past medical images (hereinafter referred to as “past images”) from the report server 7. The current image and the past image are images of the same subject as an imaging target. The current image is an example of a first image of the present disclosure, and the past image is an example of a second image of the present disclosure.

The extraction unit 32 extracts the image finding information indicating at least one type of findings included in the current image by executing a plurality of types of finding extraction processes (see FIG. 8) on the current image. Specifically, the extraction unit 32 executes the finding extraction processes M1 to M6 on each of the plurality of tomographic images acquired as the current image, and extracts image finding information from each tomographic image. In the following description, it is assumed that the result shown in FIG. 11 is obtained as the extraction result of the image finding information extracted by the extraction unit 32.

In addition, the extraction unit 32 extracts the document finding information included in the interpretation report describing the past image acquired by the acquisition unit 30. In the following description, it is assumed that the interpretation report describing the past image is the interpretation report of FIG. 6 and the result shown in FIG. 7 is obtained as the extraction result of the document finding information extracted by the extraction unit 32.

The specifying unit 34 specifies a finding extraction process for extracting, from the current image, image finding information indicating a finding indicated by the document finding information extracted from the interpretation report describing the past image by the extraction unit 32, among the plurality of types of finding extraction processes determined in advance.

Further, the specifying unit 34 associates the extraction result of the document finding information extracted by the extraction unit 32 with the image finding information extracted by the extraction unit 32 for the same region of interest. Specifically, the specifying unit 34 may associate the extraction result of the document finding information extracted by the extraction unit 32 with the extraction result of the image finding information obtained by executing the finding extraction process of the same type as the finding extraction process specified for the document finding information on the current image. As described above, the finding extraction processes M1 to M6 have different organs and/or lesions to be extracted. Therefore, it can be said that the document finding information and the image finding information related to the same type of finding extraction process are related to the same region of interest (that is, the organ and/or the lesion).

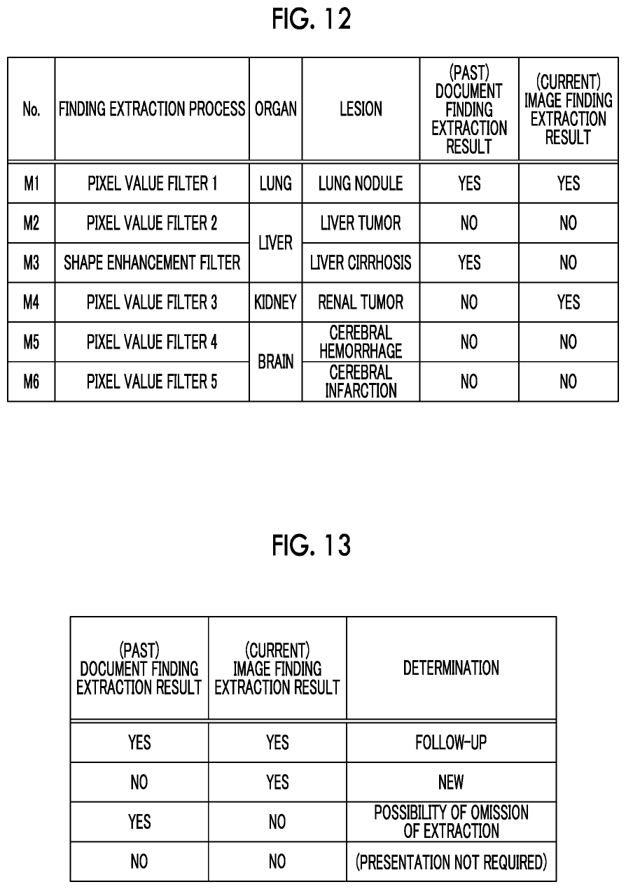

For example, the “pixel value filter 1” (finding extraction process M1) is specified for the document finding information indicating the “nodule” of the “lung”. In this case, the specifying unit 34 associates the extraction result of the document finding information indicating the “nodule” of the “lung” with the extraction result of the image finding information obtained by executing the “pixel value filter 1” on the current image. Further, for example, the “shape enhancement filter” (finding extraction process M3) is specified for the document finding information indicating the “liver cirrhosis” of the “liver”. In this case, the specifying unit 34 associates the extraction result of the document finding information indicating the “liver cirrhosis” of the “liver” with the extraction result of the image finding information obtained by executing the “shape enhancement filter” on the current image. FIG. 12 shows a result of associating the extraction result of the document finding information shown in FIG. 7 with the extraction result of the image finding information shown in FIG. 11.

In addition, the specifying unit 34 determines a pattern according to a finding included in both the extraction result of the document finding information and the extraction result of the image finding information, and a finding included in any one of the extraction result of the document finding information and the extraction result of the image finding information. FIG. 13 shows each pattern. As shown in FIG. 13, the specifying unit 34 determines that a finding included in the extraction result of the document finding information and included in the extraction result of the image finding information is a follow-up finding. In addition, the specifying unit 34 determines that a finding that is not included in the extraction result of the document finding information and is included in the extraction result of the image finding information is a new finding. In addition, the specifying unit 34 determines that a finding that is included in the extraction result of the document finding information and is not included in the extraction result of the image finding information is a finding having a possibility of omission of extraction by the finding extraction process executed by the extraction unit 32.

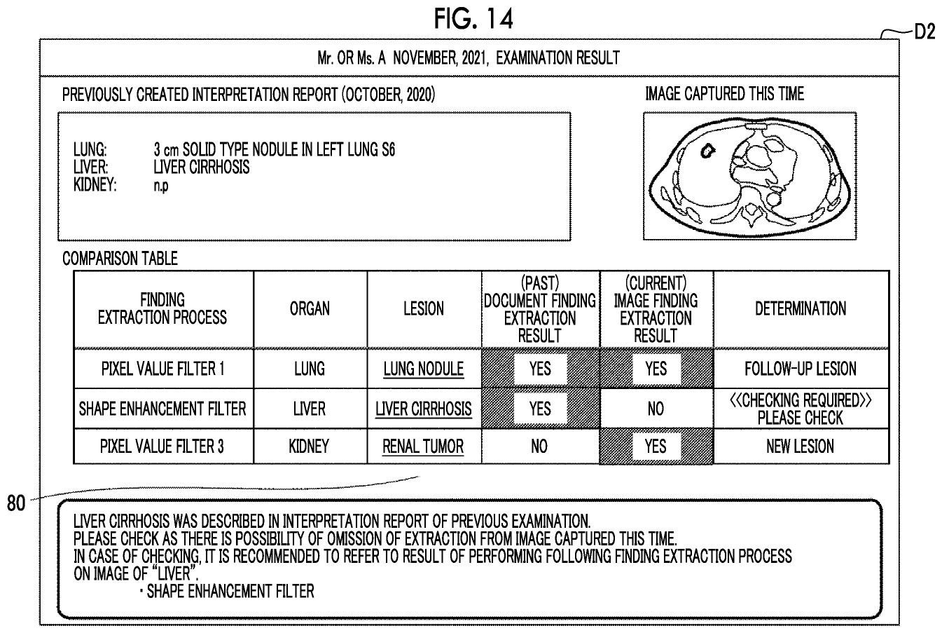

The controller 36 presents the finding extraction process specified by the specifying unit 34 with respect to the document finding information extracted from the interpretation report describing the past image by the extraction unit 32. In addition, the controller 36 presents a finding included in both the extraction result of the document finding information and the extraction result of the image finding information, and a finding included in any one of the extraction result of the document finding information and the extraction result of the image finding information in an identifiable manner. As shown in FIG. 14, for example, the expression “presenting in an identifiable manner” may be realized by displaying character strings such as a “follow-up lesion” for the follow-up finding, a “new lesion” for the new finding, and “checking required” for findings having a possibility of omission of extraction, according to the pattern determined by the specifying unit 34. In addition, for example, it may be realized by changing, according to each pattern, a display form such as a character type (color, font, bold, italic, etc.), a background color, and a type of a frame line in a case of presenting the findings. Also, for example, it may be realized by displaying an icon meaning each pattern.

FIG. 14 is an example of a screen D2 displayed on the display 24 by the controller 36. The screen D2 includes an interpretation report describing past images acquired by the acquisition unit 30 and a current image. Also, the extraction result of the document finding information and the extraction result of the image finding information extracted by the extraction unit 32 and the finding extraction process specified for the document finding information by the specifying unit 34 are presented in association with each other. Further, the determination result of the pattern by the specifying unit 34 is presented.

In addition, the controller 36 may add a hyperlink 80 to the medical image from which the image finding information is extracted to a character string indicating the findings (for example, “lung nodule”, “liver cirrhosis”, and “renal tumor”). The user operates a cursor (not shown) on the screen D2 via the input unit 25, and selects a character string to which the hyperlink 80 is added in a case where he/she desires to view the medical image, thereby making a viewing request. For example, in a case where the hyperlink 80 added to the character string “lung nodule” is selected on the screen D2 of FIG. 14, the controller 36 may perform control such that the medical image from which the image finding information indicating the lung nodule is extracted by the extraction unit 32 is displayed on the display 24.

However, in the case of the finding that is not included in the image finding information and has a possibility of omission of extraction, the medical image from which the image finding information is extracted cannot be specified. In this case, the controller 36 may use a medical image including an organ from which the finding can be extracted as a link destination of the hyperlink 80. For example, in a case where the hyperlink 80 added to the character string “liver cirrhosis” is selected on the screen D2 of FIG. 14, the controller 36 may perform control such that a medical image including the liver is displayed on the display 24 regardless of whether or not the liver cirrhosis is extracted.

In addition, the controller 36 may automatically execute the corresponding finding extraction process on the medical image as the link destination of the hyperlink 80. For example, in a case where the hyperlink 80 added to the character string “lung nodule” is selected on the screen D2 of FIG. 14, the controller 36 may perform control such that after executing the “lung nodule extraction” (finding extraction process M1) on the medical image from which the image finding information indicating the lung nodule is extracted by the extraction unit 32, the medical image is displayed on the display 24.

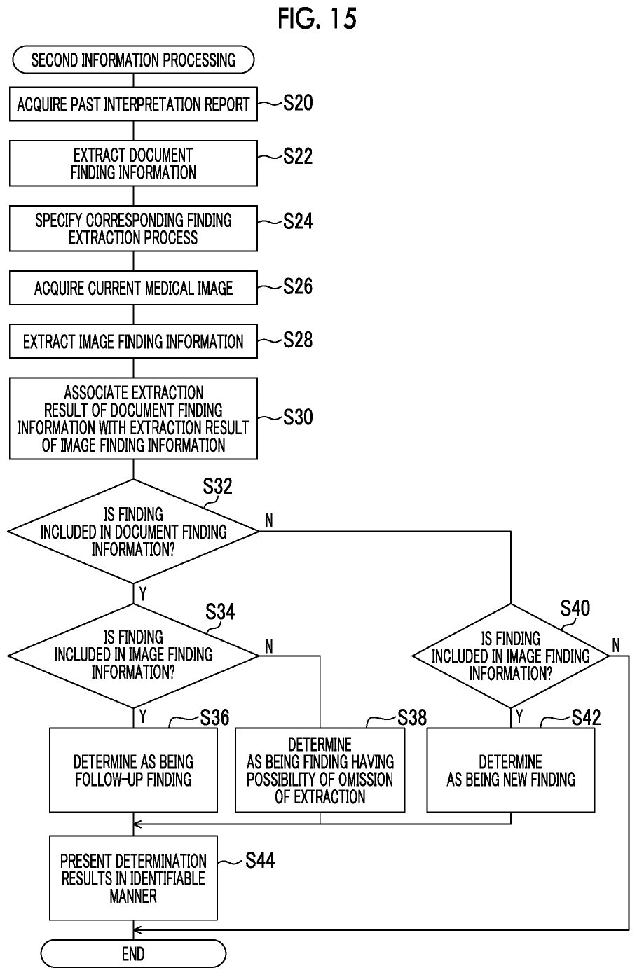

Next, with reference to FIG. 15, operations of the information processing apparatus 10 according to the present embodiment will be described. In the information processing apparatus 10, the CPU 21 executes the information processing program 27, and thus second information processing shown in FIG. 15 is executed. The second information processing is executed, for example, in a case where the user gives an instruction to start execution via the input unit 25.

In Step S20, the acquisition unit 30 acquires an interpretation report describing the past image from the report server 7. In Step S22, the extraction unit 32 extracts the document finding information included in the interpretation report acquired in Step S20. In Step S24, the specifying unit 34 specifies the finding extraction process for extracting the image finding information indicating the finding indicated by the document finding information extracted in Step S22, among the plurality of types of finding extraction processes determined in advance.

In Step S26, the acquisition unit 30 acquires a current image from the image server. In Step S28, the extraction unit 32 extracts the image finding information indicating at least one type of findings included in the current image acquired in Step S26 by executing the plurality of types of finding extraction processes on the current image. In Step S30, the specifying unit 34 associates the extraction result of the document finding information extracted in Step S22 with the extraction result of the image finding information extracted in Step S28.

In Steps S32 to S42, the specifying unit 34 determines a pattern according to a finding included in both the extraction result of the document finding information and the extraction result of the image finding information and a finding included in any one of the extraction result of the document finding information and the extraction result of the image finding information based on the extraction result of document finding information and the extraction result of image finding information associated in step S30. Specifically, the specifying unit 34 determines that the finding included in the extraction result of the document finding information (Yin Step S32) and included in the extraction result of the image finding information (Yin Step S34) is a follow-up finding, as shown in Step S36. In addition, the specifying unit 34 determines that the finding that is included in the extraction result of the document finding information (Y in Step S32) and is not included in the extraction result of the image finding information (N in Step S34) is a finding having a possibility of omission of extraction, as shown in Step S38. In addition, the specifying unit 34 determines that the finding that is not included in the extraction result of the document finding information (N in Step S32) and is included in the extraction result of the image finding information (Yin Step S40) is a new finding, as shown in Step S42.

In Step S44, the controller 36 presents the determination results of Steps S36, S38, and S42 in an identifiable manner, and ends the second information processing. On the other hand, the controller 36 does not present the determination result for the finding that is not included in the document finding information (N in Step S32) and is not included in the image finding information (N in Step S40), and directly ends the second information processing.

As described above, the information processing apparatus 10 according to one aspect of the present disclosure comprises at least one processor, in which the processor acquires a document describing a subject and extracts document finding information indicating a finding of the subject included in the document. In addition, the processor extracts the image finding information indicating at least one type of findings included in a first image obtained by imaging the subject, and associates an extraction result of the document finding information for the same region of interest with an extraction result of the image finding information. In addition, the processor presents a finding included in both the extraction result of the document finding information and the extraction result of the image finding information, and a finding included in any one of the extraction result of the document finding information and the extraction result of the image finding information in an identifiable manner.

That is, with the information processing apparatus 10 according to the present embodiment, for each finding, it is possible to present whether or not there is a description in the interpretation report describing the past image, and whether or not extraction via CAD was performed from the current image in an identifiable manner. Thereby, the radiologist can perform the interpretation work while grasping whether each finding is a follow-up finding, a new finding, or a finding having a possibility of omission of extraction via CAD. Therefore, with the information processing apparatus 10 according to the present embodiment, it is possible to support interpretation of a medical image.

Further, in the information processing apparatus 10 according to the present embodiment, it is specified whether or not the past image includes findings based on the interpretation report, and the past image is not analyzed via CAD. That is, with the information processing apparatus 10 according to the present embodiment, the findings can be compared between the past image and the current image even though the analysis of the past image via CAD is not executed, and thus the interpretation of the medical image can be supported.

In addition, although the example of the form in which the interpretation report describing the past image is applied has been described in the second embodiment, the interpretation report describing the current image can also be applied. Even in this case, the specifying unit 34 can specify the findings that are included in the document finding information and are not included in the image finding information as findings having a possibility of omission of extraction by the finding extraction process.

In addition, in the second embodiment, the form in which the extraction result of the document finding information is associated with the extraction result of the image finding information obtained by executing the finding extraction process of the same type as the finding extraction process specified for the document finding information on the current image has been described, but the method of association is not limited thereto. For example, the specifying unit 34 may associate the extraction result of the document finding information with the extraction result of the image finding information for each lesion based on the measured values such as the properties and sizes of the lesions and the information indicating the position.

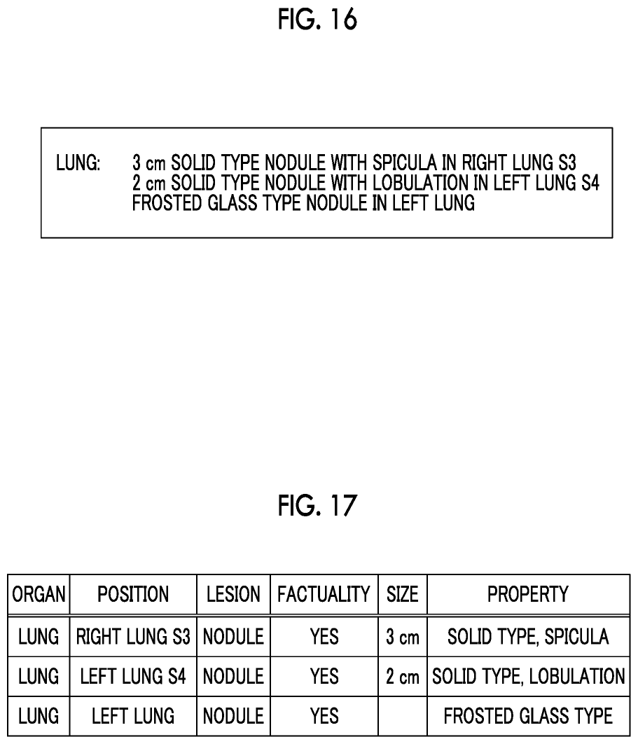

A specific example of the process of associating the extraction result of the document finding information with the extraction result of the image finding information for each lesion will be described with reference to FIGS. 16 to 19. FIG. 16 is an example of an interpretation report describing past images acquired by the acquisition unit 30, and includes descriptions regarding a plurality of lung nodules. FIG. 17 shows document finding information extracted by the extraction unit 32 from the interpretation report of FIG. 16. As shown in FIG. 17, the extraction unit 32 may distinguish lesions by using words indicating different features for each lesion, such as properties (“solid type” or the like), size (“3 cm” or the like), and position (“right lung S3” or the like).

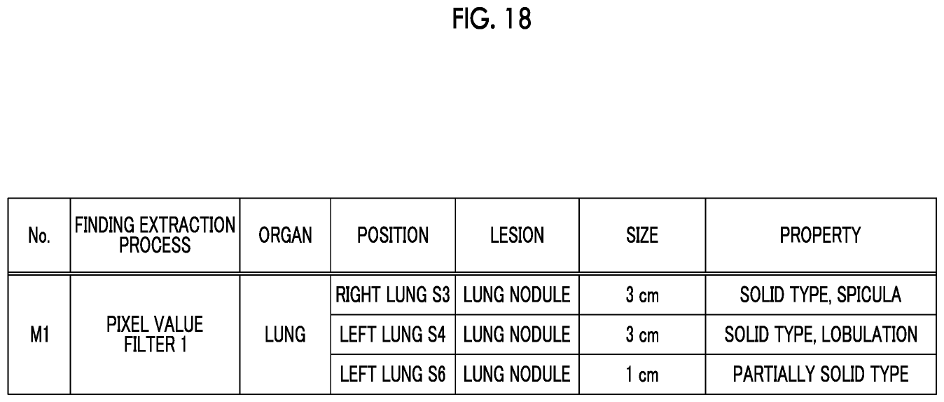

FIG. 18 shows image finding information extracted by the extraction unit 32 from the current image. As shown in FIG. 18, the extraction unit 32 may distinguish the lesions by extracting features different for each lesion, such as properties, sizes, and positions of the lesions included in the current image, from the current image.

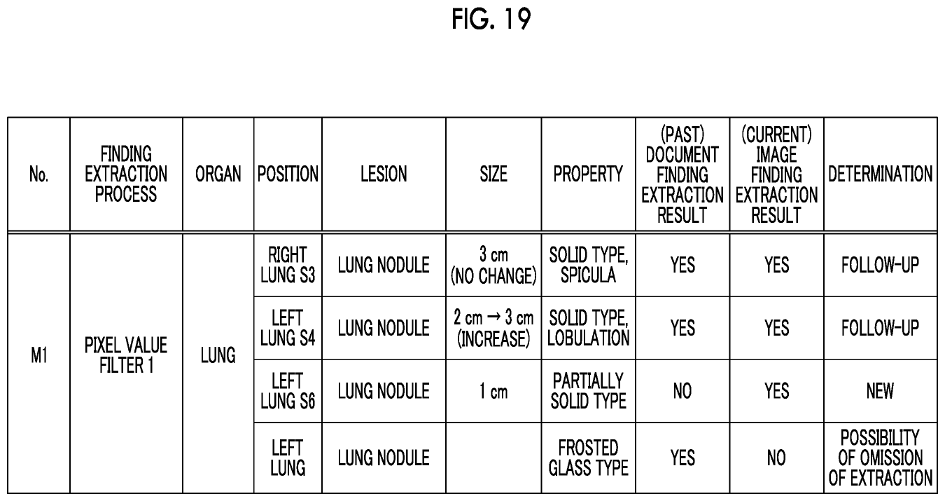

FIG. 19 shows a result of associating the extraction result of the document finding information shown in FIG. 17 with the extraction result of the image finding information shown in FIG. 18 for each lesion. As shown in FIG. 19, the specifying unit 34 may associate the extraction result of the document finding information in which at least one of the findings indicating the property, size, and position of the lesion matches with the extraction result of the image finding information. In addition, the specifying unit 34 may perform pattern determination of the finding included in both the extraction result of the document finding information and the extraction result of the image finding information, and the finding included in any one of the extraction result of the document finding information and the extraction result of the image finding information for each lesion.

Further, the specifying unit 34 may specify a change tendency regarding a property, a size, and a position of a lesion determined to be a follow-up lesion. The “change tendency” is, for example, improvement or deterioration of properties, enlargement or reduction of lesion size, primary disease and metastasis of lesion, degree of these changes (large/small/no change), and the like. In the example of FIG. 19, for the lesion whose size has increased from “2 cm” to “3 cm”, information indicating a change tendency of “increase” is added to the field of “size”. The controller 36 may present information indicating this change tendency.

Further, in each of the above embodiments, the form in which the medical image is used as an example of the first image and the second image has been described, but the technique of the present disclosure can also use an image other than the medical image. For example, the technique of the present disclosure can be applied to images (for example, CT images, visible light images, infrared images, and the like) captured in non-destructive inspection of civil engineering structures, industrial products, pipes, and the like and reports describing the images.

In the above embodiments, for example, as hardware structures of processing units that execute various kinds of processing, such as the acquisition unit 30, the extraction unit 32, the specifying unit 34, and the controller 36, various processors shown below can be used. As described above, the various processors include a programmable logic device (PLD) as a processor of which the circuit configuration can be changed after manufacture, such as a field programmable gate array (FPGA), a dedicated electrical circuit as a processor having a dedicated circuit configuration for executing specific processing such as an application specific integrated circuit (ASIC), and the like, in addition to the CPU as a general-purpose processor that functions as various processing units by executing software (program).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, as represented by a system on chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the above embodiment, the information processing program 27 is described as being stored (installed) in the storage unit 22 in advance; however, the present disclosure is not limited thereto. The information processing program 27 may be provided in a form recorded in a recording medium such as a compact disc read only memory (CD-ROM), a digital versatile disc read only memory (DVD-ROM), and a universal serial bus (USB) memory. In addition, the information processing program 27 may be downloaded from an external device via a network. Further, the technique of the present disclosure extends to a storage medium for storing the information processing program non-transitorily in addition to the information processing program.

The technique of the present disclosure can be appropriately combined with the above-described embodiments. The described contents and illustrated contents shown above are detailed descriptions of the parts related to the technique of the present disclosure, and are merely an example of the technique of the present disclosure. For example, the above description of the configuration, function, operation, and effect is an example of the configuration, function, operation, and effect of the parts according to the technique of the present disclosure. Therefore, needless to say, unnecessary parts may be deleted, new elements may be added, or replacements may be made to the described contents and illustrated contents shown above within a range that does not deviate from the gist of the technique of the present disclosure.

What is claimed is:

1. An information processing apparatus comprising at least one processor, wherein the at least one processor is configured to:
acquire a document describing a subject;
extract document finding information indicating a finding of the subject included in the document; and
specify a finding extraction process for extracting image finding information indicating the finding indicated by the document finding information from a first image obtained by imaging the subject, among a plurality of types of finding extraction processes for extracting image finding information indicating a plurality of different types of findings that are able to be included in the first image.

2. The information processing apparatus according to claim 1, wherein the at least one processor is configured to:
acquire the first image; and
extract the image finding information indicating at least one type of findings included in the first image by executing the plurality of types of finding extraction processes on the first image.

3. The information processing apparatus according to claim 2, wherein the at least one processor is configured to associate an extraction result of the document finding information for the same region of interest with an extraction result of the image finding information.

4. The information processing apparatus according to claim 2, wherein the at least one processor is configured to associate an extraction result of the document finding information with an extraction result of the image finding information obtained by executing the finding extraction process of the same type as the finding extraction process specified for the document finding information on the first image.

5. The information processing apparatus according to claim 3, wherein the at least one processor is configured to present a finding included in both the extraction result of the document finding information and the extraction result of the image finding information, and a finding included in any one of the extraction result of the document finding information and the extraction result of the image finding information in an identifiable manner.

6. The information processing apparatus according to claim 5, wherein the at least one processor is configured to make presentation indicating a possibility of omission of extraction by the finding extraction process for a finding that is included in the extraction result of the document finding information and is not included in the extraction result of the image finding information.

7. The information processing apparatus according to claim 5, wherein:
the document is a document describing a second image obtained by imaging the subject at an imaging point in time prior to an imaging point in time of the first image, and
the at least one processor is configured to make presentation indicating being followed up for a finding that is included in the extraction result of the document finding information and is included in the extraction result of the image finding information.

8. The information processing apparatus according to claim 5, wherein:
the document is a document describing a second image obtained by imaging the subject at an imaging point in time prior to an imaging point in time of the first image, and
the at least one processor is configured to make presentation indicating a new finding for a finding that is not included in the extraction result of the document finding information and is included in the extraction result of the image finding information.

9. The information processing apparatus according to claim 1, wherein the document is a document describing a second image obtained by imaging the subject at an imaging point in time prior to an imaging point in time of the first image.

10. The information processing apparatus according to claim 1, wherein:
the first image is a medical image,
the document finding information and the image finding information are information indicating at least one of a name, a property, a measured value, a position, or an estimated disease name related to a region of interest included in the medical image, and
the region of interest is at least one of a region of a structure included in the medical image or a region of an abnormal shadow included in the medical image.

11. An information processing method comprising:
acquiring a document describing a subject;
extracting document finding information indicating a finding of the subject included in the document; and
specifying a finding extraction process for extracting image finding information indicating the finding indicated by the document finding information from a first image obtained by imaging the subject, among a plurality of types of finding extraction processes for extracting image finding information indicating a plurality of different types of findings that are able to be included in the first image.

12. A non-transitory computer-readable storage medium storing an information processing program for causing a computer to execute a process comprising:
acquiring a document describing a subject;
extracting document finding information indicating a finding of the subject included in the document; and
specifying a finding extraction process for extracting image finding information indicating the finding indicated by the document finding information from a first image obtained by imaging the subject, among a plurality of types of finding extraction processes for extracting image finding information indicating a plurality of different types of findings that are able to be included in the first image.
